(19) United States
(12) Patent Application Publication
(10) Pub. No.: US 2008/0027468 A1
(43) Pub. Date: Jan. 31, 2008

(54) Suture needle, suture needle/suture assembly and suture passer device

(75) Inventors: Paul V. Fenton JR., Marblehead, MA (US); George Adaniya, Wellesley, MA (US); Thomas Egan, Marblehead, MA (US); Francis Harrington, Peabody, MA (US); Peter K. Sorensen, Salem, MA (US); Justin Anderson, Edwards, CO (US)

Correspondence Address: FOLEY & LARDNER LLP, 111 HUNTINGTON AVENUE, 26TH FLOOR, BOSTON, MA, 02199-7610, US

(73) Assignee: Axya Medical Inc.

(21) Appl. No.: 11/881,329

(22) Filed: Jul. 26, 2007

Related U.S. Application Data
(60) Provisional application No. 60/833,714, filed on Jul. 27, 2006.

(51) Int. Cl. A61B 17/04; A61B 17/06

(52) U.S. Cl. 606/144; 606/222

(57) ABSTRACT

A blade suture needle is adapted for use in a suture needle assembly, and with a suture passer device, for passing a suture needle assembly through tissue. The suture needle includes an elongated resilient sheet body member bounded by opposite principal surfaces. The body member includes at least one central aperture extending between the principal surfaces, having dimensions to permit of a capturing flange to pass therethrough. The suture needle may be part of a needle assembly further including an elongated suture passing through an aperture in the needle. A suture passer device includes an elongated pusher guide with pair of opposed jaw members disposed at its distal end, which may be opened or closed in response to a user action at its proximal end. The guide allows passage of an elongated pusher element through a pusher channel therein. One of the jaw members includes a suture assembly channel aligned with the pusher channel. The other jaw member has an aperture passing therethrough, which is aligned with the suture assembly channel when the jaw members are closed. This other jaw member includes an elongated resilient flange fixedly or retractably attached to the jaw member at the periphery of the aperture, and extending across the aperture to an opposite point on the periphery of the aperture.

### FIELD OF THE INVENTION

This invention relates generally to surgical suturing and, in particular, to improved articles, instrumentation, assemblies, and methods therefore.

### BACKGROUND

With the advent of Minimally Invasive Surgery (MIS) surgeons are increasingly called upon to suture tissue under endoscopic visualization, using tools to manipulate indirectly needles and suture. A prime example of this is arthroscopic surgery of the shoulder, where instruments must pass through a tubular cannula, then pass suture through tissue—the rotator cuff, for example—at a right angle to the axis of the cannula and instrument. One difficulty encountered is capturing the suture in a secure manner after it has passed through the tissue to enable the suture to be secured in place.

Numerous attempts have been made to facilitate MIS suture passing. U.S. Pat. No. 4,957,498 (Caspari et al.) advanced the art with a suturing instrument including a hollow needle for penetrating tissue when clamped between two jaws, and a mechanism for feeding suture through the hollow needle. More recently, U.S. Pat. No. 6,638,283 (Thal), discloses a suture passer with a hollow tube adapted to be a guide for a needle attached to a suture. Both of these inventions suffer a significant drawback when used in conventional surgical practice. It frequently is desirable to pass suture where one end of the suture is anchored or otherwise attached elsewhere in the body, such as is the case when using common screw-in suture anchors. The devices taught by Caspari and Thal employ tubular suture conveyance tubes that fully surround the suture; however, in practice, both ends of the suture must be free to disengage the instrument from the thread.

U.S. Patent Publication 2003/0065337 A1 (Topper et al.) solves this problem by employing a bendable needle used to push or pull suture through tissue. The design of the Topper instrument includes a lateral opening for release of the suture following penetration, thereby permitting one end of the suture to be anchored. While this is an improvement over prior art, there are still numerous drawbacks. First, the bendable needle is part of the instrument and is reused for multiple penetrations of tissue. Thus, the needle in the Topper invention is prone to becoming dull or fatigued from repeated bending and straightening cycles. In practice, the needle of the Topper device must be replaced every surgical case, significantly increasing the cost of the procedure. Second, the needle disclosed in Topper includes a slot in the tip adapted to capture suture. This slot has a forward facing opening in the push-suture version and a retrograde opening in the pull-suture version. When pushing or pulling through fibrous tissue, like the rotator cuff of the shoulder, this slot captures and tears tissue fibers in the vicinity of the sutured repair, weakening the tissue in the least desirable location. Because the needle is both advanced and withdrawn with every pass, there is no orientation of the slot that would not tear tissue fibers at some point of the procedure. Third, suture must be loaded into the instrument in the proper orientation for it to release from the instrument. In practice, suture frequently is loaded in the wrong orientation, resulting in the suture being trapped in the instrument and necessitating re-passing of the suture.

Sutures typically are tied onto or otherwise attached to the needle by passing the suture through an opening at the proximal end of the needle, in a manner similar to that used for a sewing needle. In MIS, a suture may include a loop along the suture length, to facilitate tying tissue together. However, in creating the loop in the suture, one end of the suture forming the loop is left exposed. The exposed suture end can cause tearing of the tissue and may catch in the tissue as the surgeon attempts to pass it through the tissue. In an endoscopic surgical space, even minor tears in tissue may have extreme deleterious effects.

Sutures may be delivered by a passer device, used for delivering suture to a desired location during surgery. A suture punch system similar to a passer device is described in U.S. Publication No. 2004/249394 A1 (Morris et al.). In that publication, the suture punch system is described as having a rigid, fixed lower jaw for delivering a malleable needle through tissue and up and through tissue held in place between the fixed lower jaw and an upper moveable jaw. The suture punch system described in Morris includes an opening in the upper jaw through which the needle passes once it passes through the tissue. The needle passes through the slot sufficient to draw the attached suture through the tissue, and the passer device, including the needle positioned in the upper jaw, is removed from the location to leave the suture in place in the tissue. This suture punch system does not solve the problem of ensuring that removal of the system will fully disengage the needle from the tissue. Although the needle is curved above the upper jaw, and by that configuration the upper jaw is intended to hold the needle in place while the suture punch system is removed, in practice the needle may be pulled back into the tissue if there is sufficient resistance provided by the tissue. In an MIS environment in particular, ineffectual capturing of the needle may lead to significant tearing of the affected tissue and damage to the surgery site. Thus, an improved system for passing a suture through tissue and securely removing a suture needle is needed.

Although advances have been made in endoscopic surgical devices, there remains a need for an effective device for reliably delivering suture to a surgery site, which ensures that a needle passed through tissue will not slip back into the tissue prior to removal. There also remains a need for needles, as well as needles having attached sutures that will minimize damage to ancillary tissue to the surgery site.

### SUMMARY OF THE INVENTION

The present invention provides a blade suture needle, which may be used in a suture needle assembly, and a suture passer device for passing a suture needle assembly through tissue and securely engaging the needle after it passes through tissue to be sutured.

According to one aspect of the present invention, the suture needle includes an elongated resilient sheet body member having a relatively thin thickness extending along a needle axis between a proximal end and a pointed distal end. The body member is bounded by opposite principal surfaces and a lateral surface that extends between the principal surfaces. The body member includes at least a central aperture disposed along the needle axis between the principal surfaces from a point P1 near the proximal end to a point P2 near the distal end. The central aperture has a width W, transverse to the needle axis near point P2, sufficient to allow a suture or a needle capturing flange to be positioned therewithin.

In one embodiment, the body member includes a central aperture and an open-ended end aperture extending along the needle axis. The open-ended end aperture is used to ensure that an attached suture remains forward of the proximal end to prevent damage to the suture when pushing the needle from the proximal end.

In another embodiment, the body member includes one or more additional apertures to be used for securing sutures, facilitating use of the needle in a needle assembly, or otherwise as necessary to use the needle in combination with other surgical instruments. In alternative embodiments, any of the central aperture and the additional apertures may be oval, elongate, round, or otherwise shaped as desired for use of the needle with surgical devices and sutures.

In addition, the present invention further includes a suture needle assembly having an elongated needle and an elongated suture. In a preferred embodiment, the elongated needle includes a body member extending along a needle axis from a proximal end to a pointed distal end. The needle includes an aperture disposed along the central axis between a point P1 relatively near the proximal end and a proximal point P2 relatively near the distal end. The suture of the preferred embodiment extends along a suture axis between a first end and a second end, whereby the first end of the suture extends through a central aperture in the needle. The suture wraps around the body member of the needle between point P and the distal end of the needle. The first end portion of the suture is joined to a second end portion of the suture at an intermediate portion of the suture.

In a preferred embodiment of the suture needle assembly of the invention, the suture needle assembly includes a blade needle. In alternative embodiments, the needle includes a conventional cylindrical needle.

In a preferred embodiment, the suture axis of the first end portion of the suture, the suture axis of the second portion, and the suture axis of the intermediate portion are substantially parallel and lie substantially in a reference plane. In alternative embodiments, the first end portion of the suture, the second end portion of the suture, and the intermediate portion of the suture are positioned in a substantially stacked manner. In another embodiment, the end portions are mutually joined by fused regions of the suture. The fused regions may be formed by applied heat, applied ultrasonic energy, or an applied bonding agent. In another embodiment, the fused regions are joined using a sleeve or other device for holding the portions of the suture in the desired position on the needle.

In addition, the present invention provides a suture passer device that includes an elongated pusher guide extending along a pusher axis between a proximal end and a distal end, a jaw assembly disposed at the distal end of the pusher guide, and an assembly for opening and closing the jaw members from points near the proximal end to the pusher guide.

In a preferred embodiment, the pusher guide includes a pusher channel therein extending along the pusher axis. The pusher channel is adapted to allow passage therethrough of a distal end of an elongated pusher element. In the preferred embodiment, the jaw assembly includes a first jaw member and a second jaw member, wherein at least one of the jaw members is pivotally coupled about a first hinge axis transverse to the pusher axis, with the jaw members including mutually facing jaw surfaces. Also in the preferred embodiment, one of the jaw members includes a suture assembly channel having a first end aligned with the pusher channel at the distal end of the pusher guide, having a second end at an exit port of the jaw surface of one of the jaw members, and having a suture port at or near the first end. The suture assembly channel is adapted to pass the suture assembly therethrough from the suture port to the second end. The other jaw member has an aperture passing therethrough, which aperture is the same distance from the hinge axis as is the exit port from the hinge axis. This other jaw member includes an elongated resilient flange fixedly attached to the jaw member at a first point near the periphery of the aperture, and extending in the direction of the jaw axis of the jaw member and across the aperture to a second point near the periphery of the aperture.

In alternative embodiments of the invention, one of either jaw member is rigidly coupled to the pusher guide whereby the jaw axis of this jaw member is parallel with the pusher axis. In another embodiment, the jaw member not rigidly coupled to the pusher guide, is pivotally coupled about a second hinge axis transverse to the pusher axis. In that embodiment, the jaw axes of both of the jaw members are angularly moveable with respect to the pusher axis in response to the opening and closing of the jaw members. The first hinge axis and the second hinge axis may be coaxial or mutually spaced apart. Alternatively, the first end of the jaw channel and the suture port are coaxial.

### BRIEF DESCRIPTION OF THE DRAWINGS

FIG. 1 is a perspective view of an embodiment of the suture needle of the present invention;

FIG. 2 is a top plan view of an embodiment of the suture needle of the present invention;

FIG. 3 is a top plan view of an alternative embodiment of the suture needle of the present invention;

FIGS. 3A-3D show perspective and top plan views of alternative embodiments of suture needle of the present invention.

FIG. 4 is a side plan view of a suture needle assembly of the present invention, showing use of the inventive suture needle with a standard prior art suture;

FIG. 5A is a side plan view of one embodiment of the suture needle assembly of the present invention, showing a stacked, overlapping suture configuration with a blade needle;

FIG. 5B is a cross-section view of a suture configuration of the present invention;

FIG. 6A is a side plan view of one embodiment of the suture needle assembly of the present invention, showing a planar suture configuration with a blade needle;

FIG. 6B is a cross-section view of a suture configuration of the present invention;

FIG. 7 is a side plan view of one embodiment of the suture needle assembly of the present invention, showing a stacked, overlapping suture configuration with a standard cylindrical needle;

FIG. 8 is a side plan view of one embodiment of the suture needle assembly of the present invention, showing a stacked, overlapping suture configuration with a standard cylindrical needle;

FIG. 9 is a perspective view of a suture passer device, including an integrated handle and a tissue and needle capturing system of the present invention;

FIG. 10 is a side plan cutaway view of one embodiment of a suture passer device having an integrated tissue and needle capturing system employing a suture needle assembly of the present invention;

FIG. 11 is a side plan cutaway view of the suture passer device of FIG. 9, showing a suture needle assembly deployed within the suture port of the system;

FIG. 12 is a side plan cutaway view of the suture passer device of FIG. 9, showing the malleable needle of the suture needle assembly passing through tissue;

FIG. 13 is a side plan cutaway view of the suture passer device of FIG. 9, showing the needle of the suture needle assembly passing through an aperture in the upper jaw portion of the tissue and needle capturing system;

FIG. 14 is a perspective view of a suture needle assembly of the present invention deployed within the aperture of the top moveable jaw of the tissue and needle capturing system;

FIG. 15 is a perspective view of the suture passer device of FIG. 14, showing the suture needle assembly captured by the tissue and needle capturing system as the suture is pulled through tissue;

FIG. 16 is a perspective view of the suture passer device of FIG. 14, showing the suture needle assembly fully captured by the tissue and needle capturing system just prior to removing the entire suture passer device from the surgery site;

FIG. 17 is a side plan cutaway view of an alternative embodiment of the suture passer device of the present invention, having a fixed upper jaw and a moveable lower jaw;

FIG. 18 is a side plan cutaway view of an alternative embodiment of the suture passer device of the present invention, having a moveable upper jaw and a moveable lower jaw;

FIG. 19 is a side plan cutaway view of an alternative embodiment of the suture passer device of the present invention, having the suture port positioned in the pusher guide portion of the device;

FIG. 20 is a top view of an embodiment of the tissue and needle capturing system of the present invention, with an embodiment of the blade needle of the present invention captured therein;

FIG. 21 is a side view of the tissue and needle capturing system of the present invention, showing the blade needle fully captured therein;

FIG. 22 is a perspective view of an alternative suture passer device of the present invention having a suture needle assembly of the present invention deployed within the aperture of the top moveable jaw of the tissue and needle capturing system;

FIG. 23 is a perspective view of the suture passer device of FIG. 22, showing the suture needle assembly captured by the tissue and needle capturing system as the suture is pulled through tissue;

FIG. 24 is a perspective view of the suture passer device of FIG. 22, showing the suture needle assembly fully captured by the tissue and needle capturing system just prior to removing the entire suture passer device from the surgery site.

Like elements in respective figures have the same reference numbers.

### DETAILED DESCRIPTION OF THE INVENTION

The present invention provides a system for reliably delivering and securing a suture passed through tissue, in particular during endoscopic surgical procedures. The invention provides a flat blade suture needle. In some forms, the needle is malleable and, in other forms, the needle is resilient. In one form, the needle assembly includes a suture, which is configured to form a loop (which acts as a “quote” eyelet) that enables a length of suture to pass through, such that the needle assembly draws the suture through the target tissue. Using the suture passer device of the present invention, in various embodiments, the needle assembly is reliably captured and removed using the integral tissue and needle capturing system, leaving the suture in place for the surgeon to further manipulate. Preferably, a tissue and needle capturing system includes a resilient flange element that securely traps or captures the needle assembly.

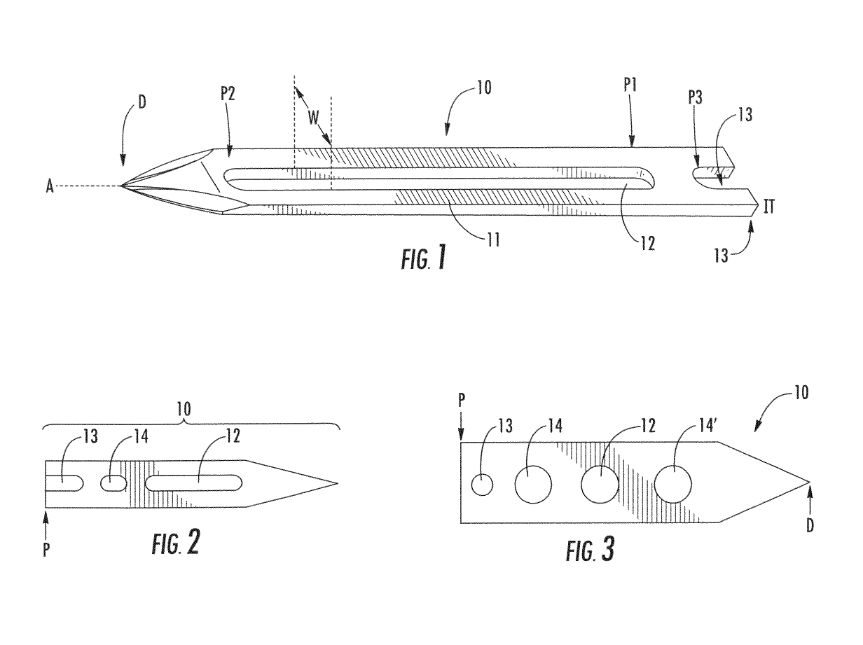

As shown in FIG. 1, the blade needle 10 of the present invention consists of a relatively flat blade body member 11 having a central aperture 12 along the needle axis A. The body member 11 preferably is manufactured from a sheet of resilient material that is resistant to damage from contact with saline, blood, and other body fluids and materials incidental to surgery. In one form, the material may be a substantially rigid material for use of the blade needle 10 in a free-standing manner or in tissue that requires substantial penetration strength. In another embodiment, the blade needle 10 may be manufactured from malleable material that enables the blade needle 10 to hold it shape after bending with a suture passer device described further below. The thickness T of the blade needle 10, as shown in FIG. 1, will vary in accordance with the required level of resilience or rigidity of the blade needle 10, the properties of the materials used to manufacture the blade needle 10, and other similar variables.

In a preferred embodiment, the thickness of the blade needle 10 is sufficient to withstand the axial loading force required to penetrate tissue, while being unsupported for a length equal to the maximum thickness of the tissue. In such a preferred embodiment, the blade needle 10 is flexible enough to bend approximately 90° around a radius less than the diameter of the delivery instrument and spring back to a straight condition. A preferred material is a superelastic material, such as Nitinol. Other less preferred materials may include stainless steel, polymer (e.g., polycarbonate, abs, nylon, acetal), or composite (e.g., fiber-based, such as carbon fiber or glass fiber).

The length of the blade needle 10 must be sufficient to fully penetrate the tissue desired to be sutured, yet short enough to fit in the available anatomical space. The width of the blade needle 10 must be sufficient to allow for suture attachment and, in a preferred embodiment, have a guiding surface area for advancement through a curved guide track, yet be narrow enough to leave a minimal hole in the tissue to be sutured to facilitate healing.

As shown, the blade needle 10 of the present invention includes a sharp leading edge having a pointed tip at a distal end D, and the central aperture 12 extends from a point P1 near the proximal end P to a point P2 near the distal end D of the blade needle 10. The distal end D may include blunt traumatic tips and other point configurations for different surgical procedures. In a preferred embodiment, the principal surfaces that form the needle 10 are planar when the needle axis A is straight, and the body member 11 is beveled at the tip of the junction of the lateral surface of one of the principal surfaces.

The central aperture 12 has a width W as may be necessary for the blade needle 10 to be used in conjunction with delivery systems, needle capture devices, and the like. In a preferred embodiment, the aperture width W is somewhat less than the width W′ of resilient flange 71 of the present invention as described further below (see FIG. 20). In alternative embodiments, the central aperture 12 is of sufficient width to receive a suture retrieval hook.

In one embodiment, and as shown in FIG. 1, the needle blade 10 may include a notch or open-ended end aperture 13 at the proximal end P of the blade needle 10 to ensure that an attached suture remains forward of the proximal end P to prevent damage to the suture when pushing the needle from the proximal end P. The end aperture 13 may be configured in a manner to facilitate use of the blade needle 10 in conjunction with a needle capture system or some other device or system requiring secure placement of the blade needle 10 for use during surgery. In a preferred embodiment, the end aperture 13 extends from for a length from the proximal end P to a point P3, which point P3 is between the proximal end P and the proximal end P1 of the central aperture. In other embodiments, the blade needle 10 does not include an end aperture 13, but may include other configurations, to facilitate use of the needle with other devices.

In one embodiment, the central aperture 12 is a single, elongate aperture extending along the needle axis A. In an alternative embodiment, and as shown in FIG. 2, the blade needle 10 includes one or more additional apertures 14 which may be round, oval, square, rectangular, or any shape as necessary to facilitate use of the blade needle 10 with other devices and equipment, including surgical equipment. In the embodiment of FIG. 2, the additional aperture 14 may be used to receive and secure a suture.

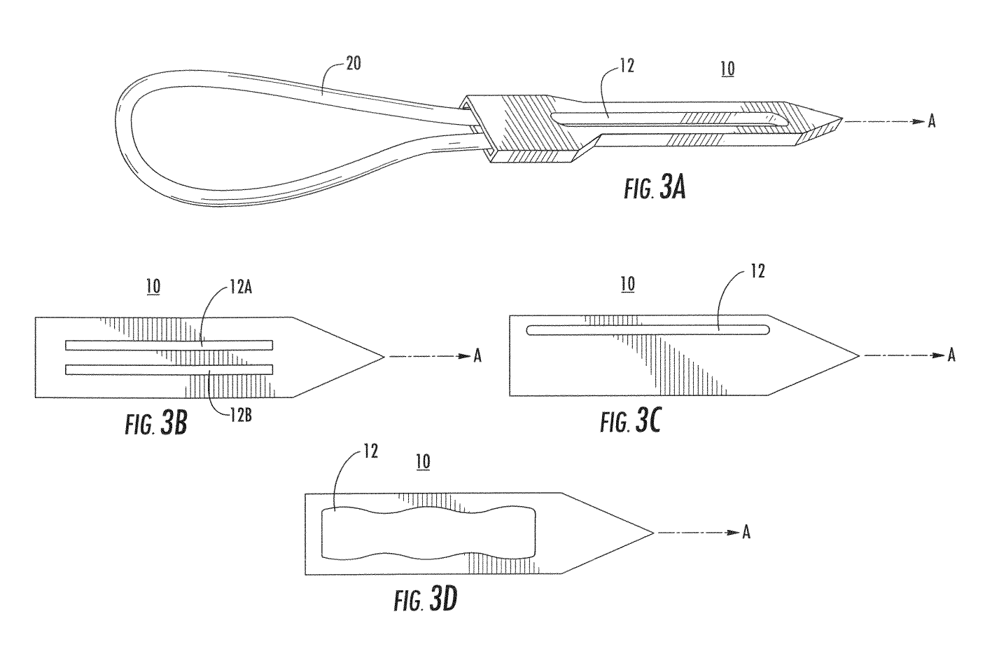

An alternative embodiment of the blade needle 10 is show in FIG. 3, wherein the central aperture 12 is one of several additional apertures 14, 14′. In that embodiment, the end aperture 13 is not open-ended, but remains proximal to the proximal end P of the needle. There are a variety of configurations of shapes, numbers, and positioning of apertures along the axis A of the blade needle 10 that may be used.

The needles of the invention can have many forms. FIGS. 3A-3D show exemplary forms for needle 10. In particular, FIG. 3A shows a perspective view of an exemplary needle 10 having an elongated central aperture 12, where the forward end of needle 10 is swaged (or flattened) from a hollow tube. A suture loop 20 has its ends captively held (for example, by crimping) by the rearward end of needle 10. FIG. 3B shows a top view of an exemplary needle 10 which is similar to the needle 10 of FIG. 1, but includes two elongated central apertures 12A and 12B extending along its principal axis A. FIG. 3C shows a top view of an exemplary needle 10 which is similar to the needle of FIG. 1, but includes a single elongated central aperture 12 which is laterally offset from its central axis A. FIG. 3D shows a top view of another exemplary needle 10 which is similar to the needle of FIG. 1, having a single elongated central aperture 12 extending along its central axis A, but where the lateral edges of the central aperture are wave-like, for example, sinusoidal. In alternative embodiments, the needles of FIGS. 3B-3D may include an end aperture, or notch, similar to end aperture 13 shown in the needle 10 of FIG. 1.

As shown in FIG. 4, the blade needle 10 of the present invention may be used in conjunction with a suture 20 attached to the blade needle 10 by the formation of an eyelet 21 at one end of the suture. The eyelet 21 is formed by threading suture 20 through aperture 14 and within end aperture 13. Alternatively, the suture 20 may be secured to the blade needle 10 using multiple passes of the suture 20 through apertures in the needle, or through passing the suture 20 through an aperture 14 with sufficient length to pass through the target tissue doubled-over.

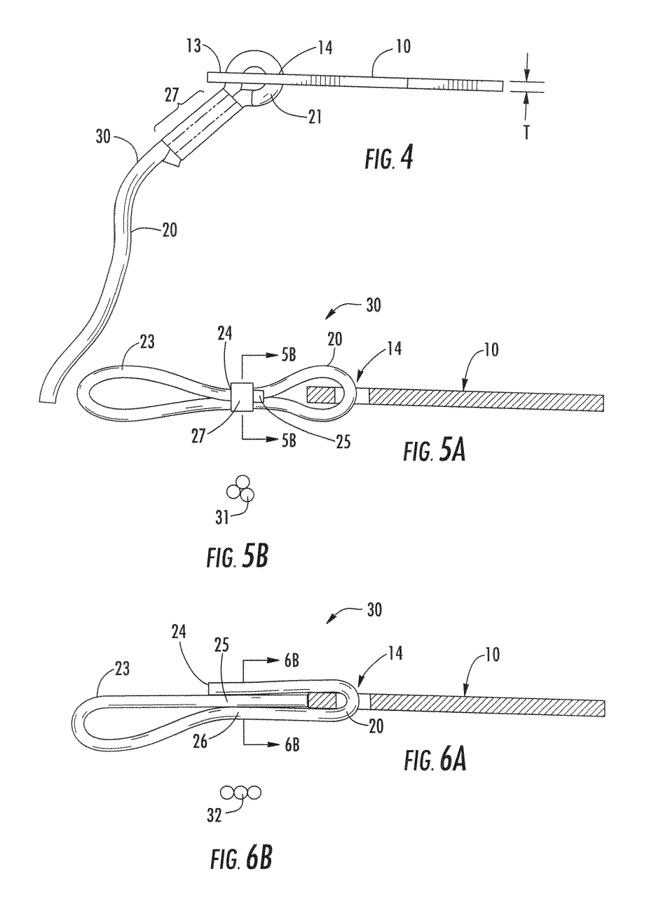

One problem encountered with using a suture 20 secured to a surgical needle in the manner described above, is that the surgeon must then cut the suture to tie it off or otherwise complete the suture procedure. As shown in FIG. 5A, the suture needle assembly 30 of this invention includes a suture 20 that is passed through an aperture 14 of a blade needle 10, then formed into a secondary suture loop 23 that extends along the needle axis. In one embodiment, as shown in the cross-section view of FIG. 5B taken along cross-section A-A of FIG. 5A, the first suture end 24 is joined at an intermediate portion 26 along the suture 20, and lies adjacent the second suture end 25 in a stacked formation, forming a stacked suture configuration 31. The suture 20 is joined at the intermediate portion 26 by suture welding, for example by ultrasonic welding or direct thermal welding such that at least adjacent portions of the intermediate portion 26 becomes fused to form a fused region 27 of the suture. Alternatively the suture 20 may be similarly positioned within an external sleeve (not shown) or by an adhesive.

In an alternative embodiment, as shown in FIG. 6A, the first suture end 24 is looped through the aperture 14 of the blade needle 10 and bent to form a suture loop 23 in a manner similar to above. However, in this embodiment, the second suture end 25 is positioned in between the first suture end 24 and an intermediate portion 26 of the loop. Thus, as shown in FIG. 6B along cross-section B-B of FIG. 6A, the suture axis of the first suture end 24, second suture end 25 and the intermediate portion 26 are substantially parallel and lie substantially in a reference plane, forming a planar suture configuration 32. In this preferred embodiment, the second suture end 25 is secured against the proximal end of the blade needle 10 such that it is not exposed to tissue during “forward” (i.e., toward the distal tip) advancement of needle 10 through tissue, thus minimizing the possibility of catching on tissue and possibly tearing tissue as the suture assembly 30 passes through tissue during surgery.

In both embodiments of the suture needle assembly 30, the fused region 27 may be formed by applied heat, ultrasonic energy, applied bonding agent, or other methods known to those in the relevant art.

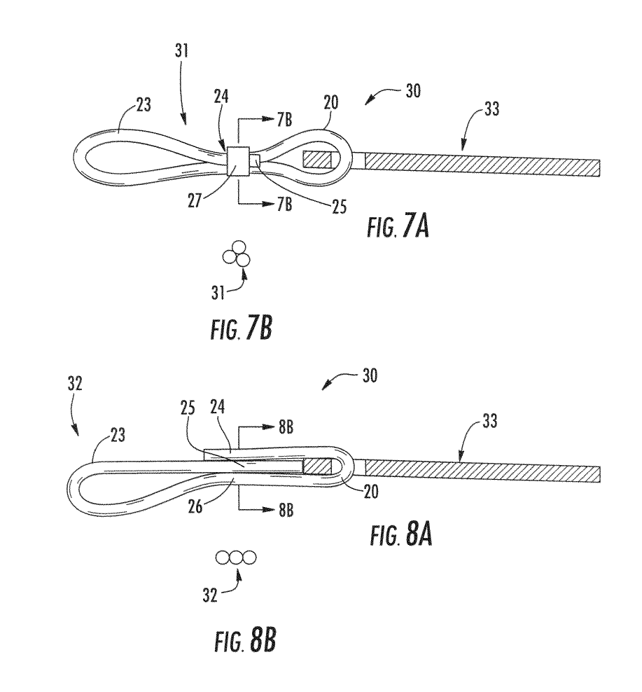

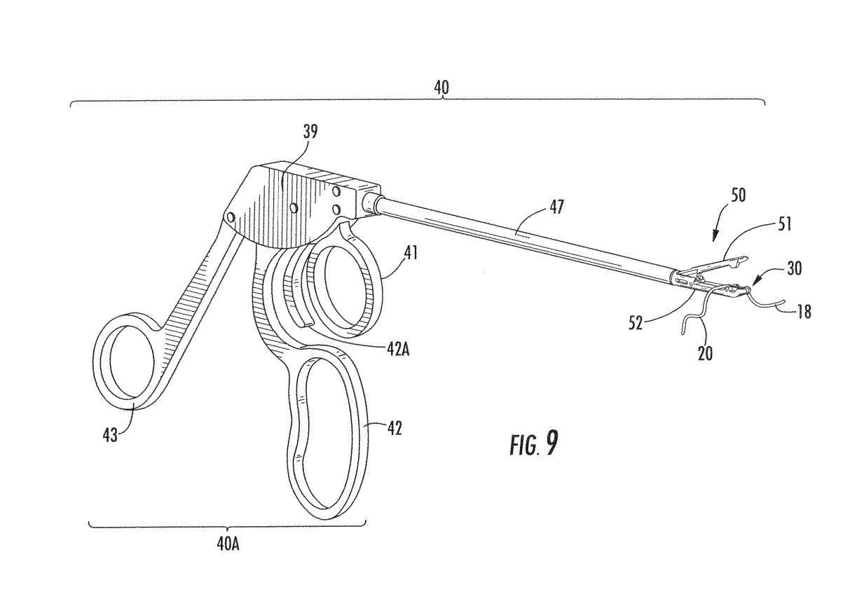

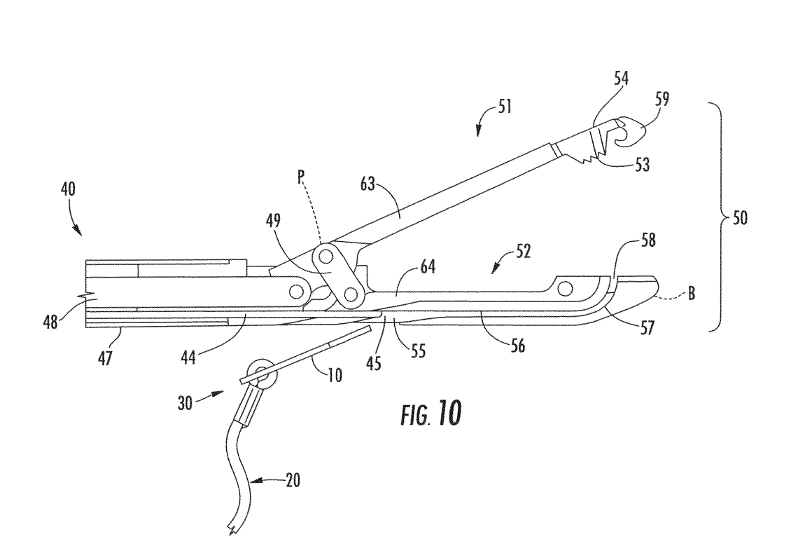

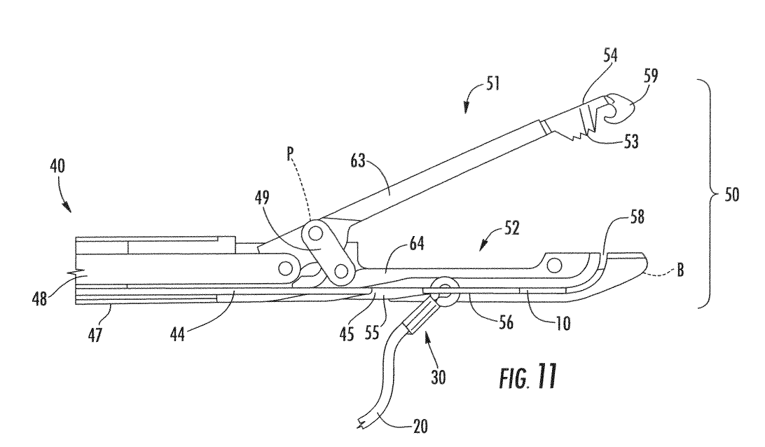

As shown in FIG. 7, the suture assembly 30 may include a standard cylindrical needle 31 together with a looped suture 23 of the type described above. Thus, one embodiment of the invention is a stacked suture configuration 31 together with a conventional cylindrical needle 33. An alternate embodiment of the suture assembly 30, as shown in FIG. 8, includes a planar suture configuration 32 together with a conventional cylindrical needle 33.

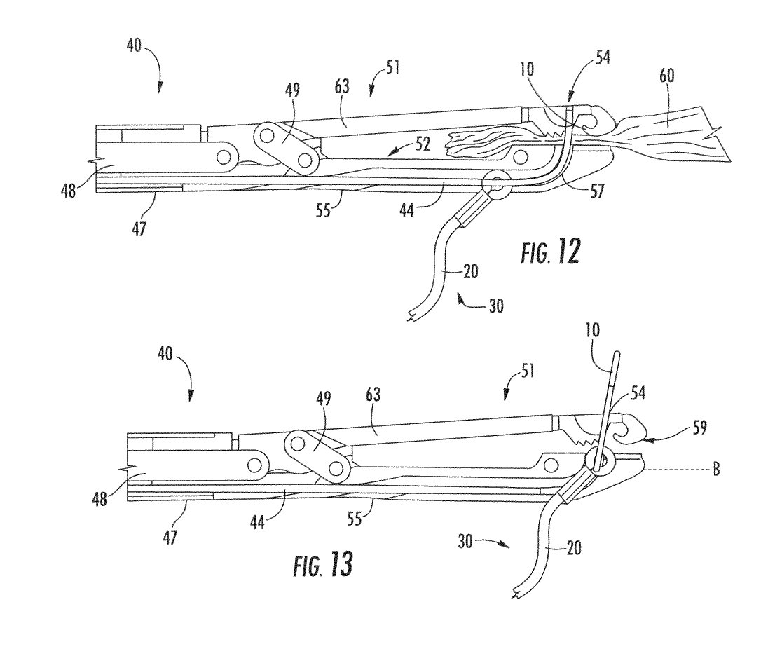

The suture needle assembly 30 of the present invention may be used in combination with and integral to a suture passer device 40, such as shown in FIG. 9. The suture passer device 40 includes a handle assembly 40A at a proximal end, and an elongated tubular element 47 extending therefrom along an axis B, and a tissue and needle capturing system 50 at a distal end. The tissue and needle capturing system 50 includes a jawed subassembly which is operable in response to user action at the handle assembly 40A to selectively open and close a set of opposed jaws 51, 52.

The proximal end of the device 40 includes a first control lever 41 for controlling pivotal movement of the upper jaw 51 about an axis (or axes) perpendicular to axis B. The proximal end of the device 40 also includes a second control lever 42 for controlling advance and retraction motion of a needle pusher (not shown in this FIG. 9). In one embodiment, as shown, the proximal end of the device further includes a needle release lever 42A for controlling the retraction of the flange 71 of the needle capturing system 70. In an alternative embodiment, the device 40 has a fixed upper jaw 51 and a moveable articulating lower jaw 52. In that embodiment, the control lever 41 may control pivotal movement of the lower jaw 52. In yet another embodiment, both upper jaw 51 and lower jaw 52 may undergo pivotal motion in response to control lever 41. The handle assembly 40A also includes a rigidly coupled base element 43 for holding the assembly 40A during use.

In addition, as shown in FIG. 10, the device 40 includes an actuating link 48 (which is coupled to control lever 41) at one end and the needle capture system 50 at the other distal end. Also shown in FIG. 10, is a pusher element 44 which is translatable within a channel within the tubular pusher guide 47.

The tissue and needle capture system 50 includes a pivotal upper jaw 51 adapted for opening and closing with respect to the lower jaw 52. In the illustrated embodiment of FIG. 10, the upper jaw 51 includes an elongate upper jaw body 63 connected at its most proximal end to the actuating link 48, and having a pivotal hinge element 49 connected to the elongated upper jaw body 63 at a point near and distal to the actuating link 48. Thus, pivotal hinge element 49 is attached at one end to the proximal end of the elongated jaw body 63 and at the other end to the elongated lower jaw body 64 to enable pivotal movement of the upper jaw 51 relative to the lower jaw 52.

The upper jaw 51 includes at the distal end, an inner rough edge element 53 that assists in securely engaging tissue to be sutured. In an alternative embodiment, either or both jaws may include the rough edge. At the distal end of the upper jaw 51 is a needle receiving aperture 54 for receiving the needle 10 of a suture needle assembly 30 from the lower jaw 52 through the upper jaw 51 during use. The needle receiving aperture 54 is positioned opposite the suture assembly aperture 58 of the lower jaw 52 for passing the suture needle assembly 30 through tissue captured between the jaws 51, 52 during operation. In one embodiment, and as illustrated in FIGS. 10-16, the distal end of the upper jaw 51 includes a retrieval hook 59 that engages the needle 10 as it passes through the needle receiving aperture 54.

The tissue and needle capture system 50 further includes a lower jaw 52 having an elongated lower jaw body 64. The proximal end of the lower jaw body 64 is pivotally attached to the pivotal hinge element 49 in a position opposite along pivotal hinge axis P. The distal end of the lower jaw body 64 includes the suture assembly aperture 58, through which the suture needle assembly 30 passes.

The device 40 further includes a substantially rigid pusher element 44 residing within a pusher channel (not shown). On the bottom of the lower jaw 52, and adjacent to and integral with the distal end of the pusher channel is a suture port 55 into which a suture needle assembly 30 may be introduced. The assembly 30 is moveable along a suture needle assembly channel 56 through the lower jaw 52. Channel 56 has an upwardly curved region 57 for guiding the suture needle assembly upward through a suture assembly aperture 58 on the uppermost surface of the lower jaw 52.

In operation, the handle assembly 40A is operable in response to a user, typically in an operation environment. A user holds the suture passer device 40 by the handle assembly 40A, typically positioning the user's thumb within the aperture of base element 43, the forefinger within the aperture of first control lever 41 and at least one additional finger in the aperture of the second control lever 42. Because the base element 43 is fixedly attached to the tissue and needle capturing system at a handle housing element 39, when the user applies pressure on the first control lever 41 by moving that lever 41 toward base element 43, it causes the actuating link 48 to move forward against the pivotal hinge element 49 and separate the jaws 51, 52. Once tissue is positioned between the jaws 51, 52, the user relieves the pressure on the first lever 41 and applies pressure on the second control lever 42 to move the pusher 44 along the pusher channel 45 and against the suture needle assembly 30 that is moved into position through the suture port and into the suture assembly channel 56. The suture needle assembly 30 is pushed out of the suture assembly aperture 58 through tissue captured between the jaws 51, 52.

FIG. 10-13 show sequential motion of the suture passer device 40 in the illustrated embodiment, as described generally above, and which now will be described in more detail.

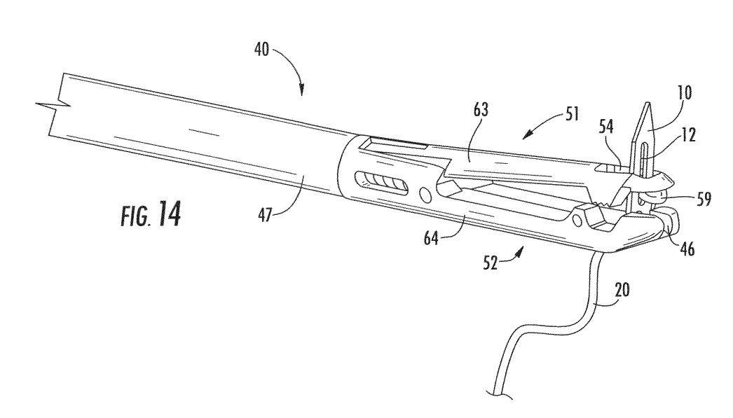

As shown in FIG. 11, the needle portion 10 of the suture needle assembly 30 is introduced into the suture port 55. Initially, the pusher element 44 is held in the pusher channel 45 until the suture needle assembly 30 is positioned within the suture needle assembly channel 56.

As shown in FIG. 12, once the suture needle assembly 30 is positioned within the suture needle assembly channel 56, the pusher element 44 is moved toward the distal end of the suture passer device 40 to occlude the suture port 55 and to apply pressure against the proximal end of the needle portion of the suture needle assembly 30. This action prevents the suture needle assembly 30 from falling back out of the suture port 55. The upper jaw 51 and lower jaw 52 then securely grasp the subject tissue 60 to be sutured. While the tissue is securely gripped between the jaws, lateral pressure is applied to the pusher element 44 against the suture needle assembly 30, to move the assembly 30 along the curved region 57 of the channel 56 and up toward the lower jaw opening 58. As the needle portion of the suture needle assembly 30 passes through the lower jaw opening 58, it pierces and traverses through the subject tissue 60. Upon passing through the tissue 60, it passes through a needle receiving aperture 54 in the upper jaw 51 which allows the entire suture needle assembly 30 to traverse through the tissue 60 to begin formation of a suture.

FIG. 13 illustrates the needle suture assembly 30 passing through the lower jaw aperture 58 and up through the needle receiving aperture 54 (the tissue is not shown in this FIG. 13 for clarity). In this illustrated embodiment, the tissue and needle capturing system includes a retrieval hook 59 that curves downward toward the needle 10. In the present invention, the suture needle assembly 30 includes a blade needle 10 of the type described above, and having a central aperture (not shown) for receivingly engaging a retrieval hook 59 as the needle 10 passes through the upper jaw aperture 54.

As illustrated in FIG. 14, as the needle 10 passes completely through the tissue (not shown), the retrieval hook 59 moves into position within the central aperture 12 of the needle 10. Then, as shown in FIG. 15, the upper jaw 51 moves upward, separating the upper jaw 51 and lower jaw 52 and in so doing, pulling the suture needle assembly 30 through the tissue (not shown). At the same time, the retrieval hook 59 engages within the aperture 12 to securely pull the suture needle assembly 30 through the tissue. Once the needle 10 is fully released from the lower jaw aperture 58, the suture 20 is free to exit the lower jaw 52 through the suture clearance passage 46. The suture clearance passage 46 may be an open channel running between the lower jaw aperture 58 and the distal end of the lower jaw 52 to enable the suture 20 to pass through the tissue to be sutured without getting caught in the tissue and needle capturing system 50.

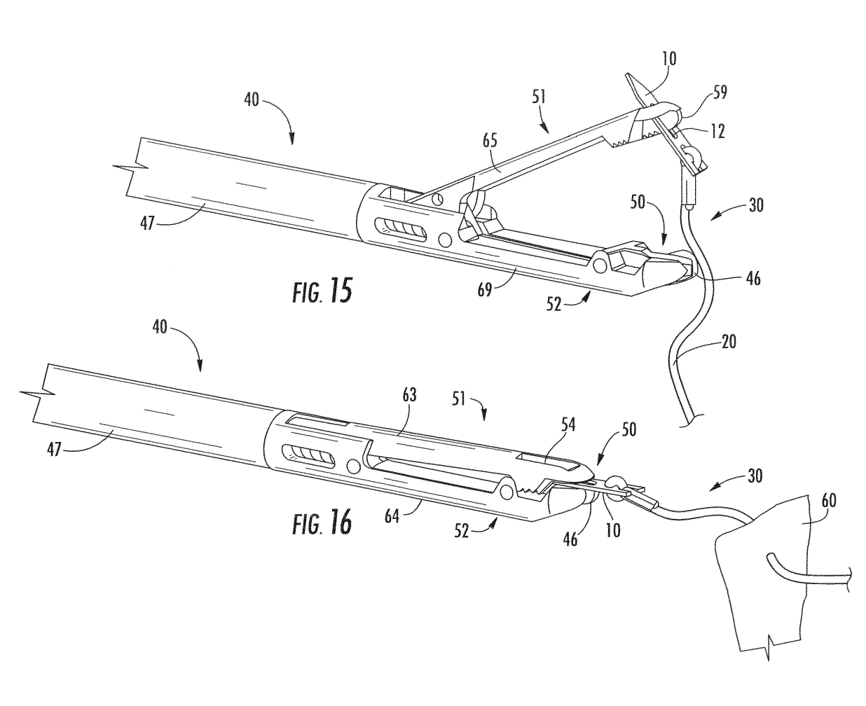

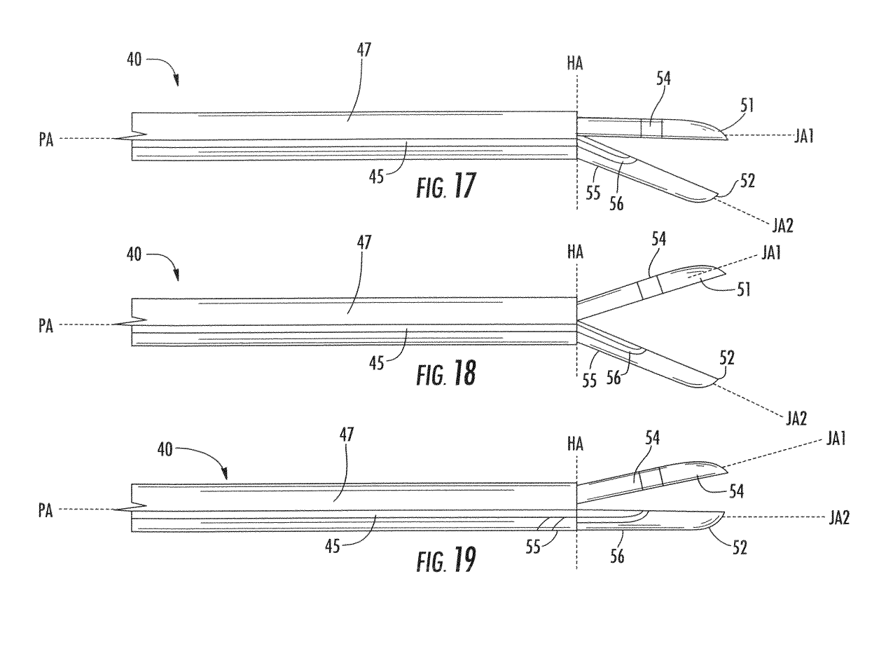

FIG. 16 shows the suture passer device 40 pulled clear of the tissue 60, with the suture 20 in position in the tissue 60 being sutured. As shown, the tissue and needle capturing system 50 is in a closed position with the needle 10 secured in the tissue and needle capturing system 50.

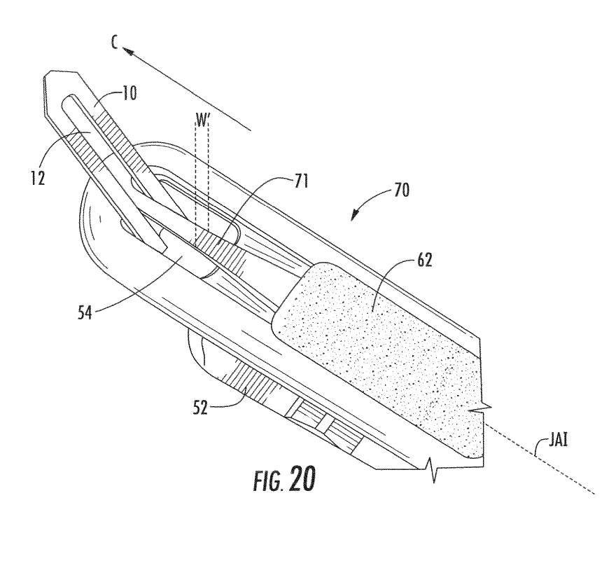

FIG. 17 shows an alternative embodiment of the suture passer device 40 of the present invention. In that embodiment, the device 40 includes a pusher guide 47 and a pusher channel 45 within which a pusher (not shown) may be introduced and used in the manner described above. In the illustrated embodiment of FIG. 17, the upper jaw 51 is fixedly attached to (or may be an extension of) the pusher guide 47, while the lower jaw 52 is pivotally coupled about a first hinge axis HA transverse to the pusher axis PA. The lower jaw 52 thus is able to move in a direction away from the upper jaw 51 to perform the suture needle assembly capturing described above. In this illustrated embodiment, the lower jaw 52 includes a suture port 55 adjacent to and integral with a suture assembly channel 56 through which a suture needle assembly may pass. The upper jaw 51 retains the needle receiving aperture 54. As illustrated, the upper jaw 51 extends along the upper jaw axis JA1, whereas the lower jaw 52 extends along a lower jaw axis JA2.

FIG. 18 shows an alternative embodiment of the suture passer device 40 of the present invention. In that embodiment, both the upper jaw 51 and the lower jaw 52 are moveable apart from each other to enable clamping of the target tissue therebetween. Both the suture port 55 and the suture assembly channel 56 are positioned in the lower jaw 52.

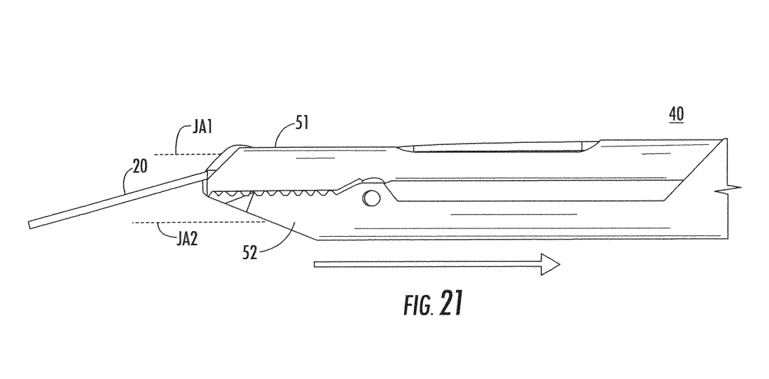

FIG. 19 shows yet another alternative embodiment of the suture passer device 40 of the present invention. In that embodiment, the lower jaw 52 is fixedly attached to the pusher guide 47. However, the suture port 55 is positioned on the lower portion of the pushed guide 47 and remains integral with a suture assembly channel 56, yet not on the lower jaw 52.

FIG. 20 shows a detailed view of preferred embodiment of the needle capturing system 70 of the present invention. As shown, the system 70 includes a resilient flange 71 attached at one end to a jaw plate 62 which plate 62 is, in a preferred embodiment, fixedly attached to the outer surface of the upper jaw 51. The flange 71 has a width W′ that is less than the width W″ of the needle receiving aperture 54. In alternative embodiments, the jaw plate 62 may be integral with the outer surface of either the upper jaw 51 or the lower jaw 52, depending upon the specific configuration of the tissue and needle capturing system 50. As shown, the flange 71 extends across the needle receiving aperture 54 through which the needle 10 passes in use. In practice, as the needle 10 passes through the needle receiving aperture 54, it applies pressure against the flange 71, moving the flange 71 away from the aperture 54. Once the needle 10 passes through a distance equal to the distance from the tip of the needle 10 to the distal end of the central aperture 12, noted as “L” in FIG. 20, the flange 71 drops through the central aperture 12, allowing the needle to pass through the needle receiving aperture 54 and simultaneously allowing the flexible flange 71 to drop into position across the needle receiving aperture 54 to secure the needle 10 within the needle receiving aperture 54. Thus secured, as shown in FIG. 21, the upper jaw 51 and lower jaw 52 can close around the suture needle assembly (not shown) and the suture passer device 40 may be pulled away from the target tissue (not shown) to be sutured.

The flange element 71 may be made from any substantially resilient material, such as stainless steel. In a preferred embodiment, such material is biocompatible or otherwise coated with a biocompatible substance so as to minimize an allergic or other undesirable response by the patient's immune system in response to introduction of the flange 71. The entire suture passer device 40 preferably is treated in a similar manner.

In some situations, a surgeon or user of the suture passer device of the invention might desire to remove the suture passer device from the surgical field after passing a needle through tissue, while leaving the captured needle in the surgical field. To accommodate that situation, a variant of the needle capturing system 70 is used. In that form of the invention, the suture passer device 40 is constructed with resilient flange 71 being selectively retractable in the direction of the longitudinal axis of upper jaw 51, by a surgeon or user.

In a first mode of operation, where a needle is being passed through tissue by device 40, the resilient flange 71 is positioned along axis C as shown in FIG. 20, and operates as described above to capture a needle 10. In a second mode of operation, where the surgeon or user of device 40 wishes to remove suture passer device 40 from the surgical field after releasing the captured needle, the resilient flange 71 is retracted in the direction of the axis C toward the proximal end of device 40, releasing the captured needle. The structure for the selective retraction of resilient flange 71 is indicted in FIG. 9 by needle release lever 42A connecting flange 71 by way of an internal link.

By retracting the resilient flange 71, after the needle 10 has been captured, the needle 10 is released from the suture passer device 40 and remains in the surgical field while the suture passer device 40 is removed therefrom.

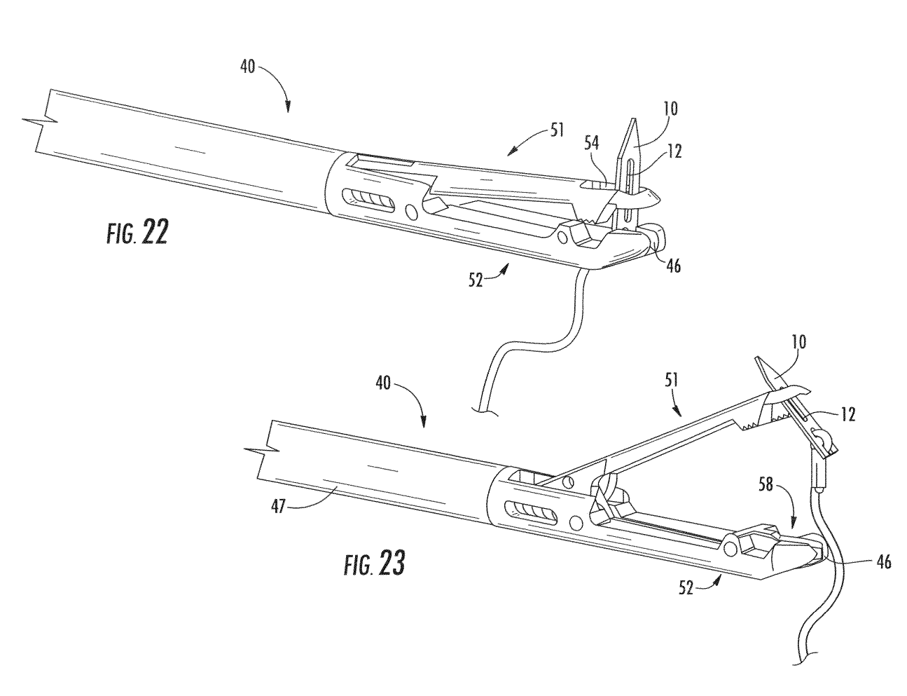

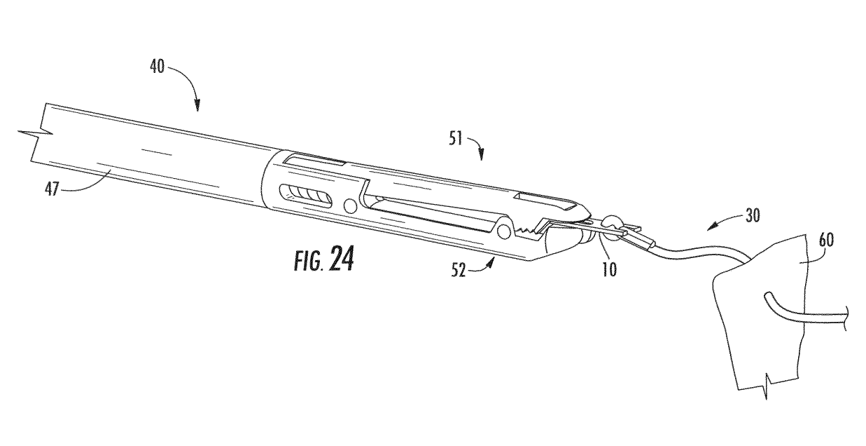

FIGS. 22-24 show a preferred embodiment of the suture passer device 40 of the present invention, having a resilient flange 71 in use to secure the needle 10 within the needle receiving aperture 54.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description. All changes that come within the meaning and range of equivalency of the claims are therefore intended to be embraced therein.

What is claimed:

1. A suture needle comprising:
an elongated resilient sheet body member having thickness T extending along a needle axis between a proximal end and a distal end, said body member being bounded by opposite principal surfaces and a lateral surface extending between said principal surfaces, and including at least one central aperture disposed along said needle axis between said principal surfaces from a point P1 near said proximal end to a point P2 near said distal and, said central aperture having a width W transverse to said needle axis near said point P2.

2. A suture needle according to claim 1 wherein said body member further includes an open-ended end aperture extending along said needle axis from said proximal end to a point P3 between said proximal end and said point P1.

3. A suture needle according to claim 1 wherein said central aperture is elongated.

4. A suture needle according to claim 3 wherein said body member further includes an open-ended end aperture extending along said needle axis from said proximal end to a point P3 between said proximal end and said point P1.

5. A suture needle according to claim 3 wherein said body member includes an end aperture extending along said central axis from a point P3 to a point P4, wherein points P3 and P4 are between said proximal end and said point P1.

6. A suture needle according to claim 1 wherein said body member includes an end aperture extending along said central axis from a point P3 to a point P4, wherein points P3 and P4 are between said proximal end and said point P1.

7. A suture needle according to claim 1 wherein said principal surfaces are planar when said needle axis is straight, and wherein body member is beveled at said tip at the junction of said lateral surface and one of said principal surfaces.

8. A suture assembly comprising:
A. an elongated needle having a body member extending along a needle axis from a proximal end to a pointed distal end, said needle having an aperture disposed along said central axis between a point P1 relatively near said proximal end and a proximal point P2 relatively near said distal end,
B. an elongated suture extending along a suture axis between a first end and a second end,
wherein a first end portion of said suture extending from said first end extends through said central aperture and wraps around said body member of said needle between point P1, and said distal end, and wherein said first end portion near said first end is joined to a second end portion of said suture extending from said second end, and
wherein said second end portion is joined to an intermediate portion of said suture, said intermediate portion being along said suture axis and between said first end portion and said second end portion,

9. A suture assembly according to claim 8 wherein said suture axis of said first end portion near said first end, said suture axis of said second portion and said suture axis of said intermediate portion are substantially parallel and lie substantially in a reference plane.

10. A suture assembly according to claim 8 wherein said end portions are mutually joined by fused regions of said suture.

11. A suture assembly according to claim 10 wherein said fused regions are formed by applied heat.

12. A suture assembly according to claim 10 wherein said fused regions are formed by applied ultrasonic energy.

13. A suture assembly according to claim 10 wherein said fused regions are formed by an applied bonding agent.

14. A suture passer device comprising:
A. an elongated pusher guide extending along a pusher axis between a proximal end and a distal end, said pusher guide including a pusher channel therein extending along said pusher axis, said pusher channel being adapted to allow passage therethrough of a distal end of an elongated pusher element;
B. a jaw assembly disposed at said distal end of said pusher guide, said jaw assembly including a first jaw member and a second jaw member, at least one of said jaw members being pivotally coupled about a first hinge axis transverse to said pusher axis, said jaw members including mutually facing jaw surfaces;
wherein each of said jaw members extends along a jaw axis from a proximal end at said hinge axis to a distal end, each of said jaw axes being transverse to said hinge axis,
wherein one of said jaw members includes a suture assembly channel having a first end aligned with said pusher channel at said distal end of said pusher guide, and having a second end at an exit port of said jaw surface of said one jaw member, and having a suture port at or near said first end, said suture assembly channel being adapted to pass said suture assembly therethrough from said suture port to said second end, said suture assembly channel being adapted to pass a suture assembly having a needle at one end coupled to an elongated suture;
wherein the other said jaw members has an aperture passing therethrough, said aperture being the same distance from said hinge axis as said exit port is from said hinge axis, and
C. means for selectively opening and closing said jaw members from points near said proximal end to said pusher guide.

15. A device according to claim 14 wherein said one jaw member is rigidly coupled to said pusher guide whereby said jaw axis of said one jaw member is parallel with said pusher axis.

16. A device according to claim 14 wherein said other jaw member is rigidly coupled to said pusher guide whereby said jaw axis of said other jaw member is parallel with said pusher axis.

17. A device according to claim 14 wherein said other of said jaw members is pivotally coupled about a second hinge axis transverse to said pusher axis, and said jaw axes of both of said jaw members are angularly movable with respect to said pusher axis in response to said opening and closing of said jaw members.

18. A device according to claim 17, wherein said first hinge axis and said second hinge axis are coaxial.

19. A device according to claim 17, wherein said first hinge axis and said second hinge axis are mutually spaced apart.

20. A device according to claim 14, wherein said first end of said jaw channel and said suture port are coaxial.

21. A device according to claim 14, wherein said other jaw includes an elongated resilient flange element fixedly attached to said other jaw member at a first point near the periphery of said aperture, and extending in the direction of said jaw axis of said other jaw member and across said aperture to second point near the periphery of said aperture, said second point being opposite said first point.

22. A device according to claim 14, wherein said other jaw includes an elongated resilient flange element retractably coupled to said other jaw member, and operable in a first mode to be at a first point near the periphery of said aperture, and extending in the direction of said jaw axis of said other jaw member and across said aperture to second point near the periphery of said aperture, said second point being opposite said first point, and operable in a second mode to be retracted in the direction of said jaw axis of said other jaw member toward said proximal end of said elongated pusher guide.
